(19) United States
(12) Patent Application Publication
(10) Pub. No.: US 2026/0096799 A1
(43) Pub. Date: Apr. 9, 2026

(54) METHOD AND APPARATUS FOR TESTING ALGORITHMS OR SYSTEMS FOR PROCESSING, EVALUATING OR RECONSTRUCTING X-RAY-BASED IMAGES

(71) Applicant: Siemens Healthineers AG, Forchheim (DE)

(72) Inventors: Fahri SIMSEKLI, Forchheim (DE); Karl SCHWARZ, Roth (DE); Gerhard LECHSEL, Erlangen (DE); Dariusz CWIEK, Warsaw (PL)

(73) Assignee: Siemens Healthineers AG, Forchheim (DE)

(21) Appl. No.: 19/344,651

(22) Filed: Sep. 30, 2025

(30) Foreign Application Priority Data
Oct. 4, 2024 (DE) 10 2024 209 722.5

(51) Int. Cl. A61B 6/58; A61B 6/03; G06T 7/00; G06T 11/00

(52) U.S. Cl. CPC A61B 6/58; A61B 6/032; G06T 7/0012; G06T 12/30; G06T 2207/10081; G06T 2207/30004; G06T 2210/41

(57) ABSTRACT

One or more example embodiments relates to a method for testing algorithms or systems for processing, evaluating or reconstructing X-ray-based images, comprising providing at least one working image, the at least one working image including a CT image; generating a number of simulation images from the at least one working image, wherein for each image point of the simulation images, image values of the at least one working image are summed along a respective line; and outputting the simulation images.

### CROSS-REFERENCE TO RELATED APPLICATION(S)

The present application claims priority under 35 U.S.C. § 119 to German Patent Application No. 10 2024 209 722.5, filed Oct. 4, 2024, the entire contents of which are incorporated herein by reference.

### FIELD

One or more example embodiments relates to a method and an apparatus for testing algorithms or systems for processing, evaluating or reconstructing X-ray-based images, a control facility for an X-ray imaging system and an X-ray imaging system.

### RELATED ART

In X-ray imaging, it is often necessary to test image reconstruction systems, image evaluation systems or image control systems for an image recording system. To do this, data from the image recording system is sent to the respective system to be tested and tests are performed. For example, raw data is sent to an image reconstruction system so that images can be reconstructed and appraised by an expert for image defects.

Typically, customer workflows in the field of computed tomography (CT) include a topo scan and a main scan. Rotation scans, static scans and sequence scans are also frequently performed; these differ in terms of their recording procedure and thus also in terms of reconstruction. Topo scans have different content than main scans. This means that there are two different sets of raw data for both scans. For realistic testing, both raw data sets must come from the same object, since the areas selected in the topo scan must exist in the main scan. If the areas do not exist or do not match, the tests cannot be performed or incorrect body areas are displayed. This means that the entire Workflow cannot be tested properly or the algorithms cannot be verified, for example, if the topo image shows the head area and the main scan only shows lung images.

If a detector does not (yet) actually exist or if patients should not be exposed to unnecessary radiation to create test data, the corresponding test data can be simulated. However, this data usually does not map reality correctly and usually does not include the correct detector behavior.

In addition, test data has to be created for each scan protocol. This can result in an extremely large amount of data. Let us assume an image reconstruction system for a new CT product type is to be tested. CT systems usually have more than 160 scan protocols and each of these protocols can be performed in multiple variants. In addition, depending on the scan, the rotation time, pitch, X-ray voltage, beam current and further parameters can be changed. This means that it is quite possible that several thousand files could be required for the product type to be tested.

If, for example, the image reconstruction system is only to be tested for a topo scan, the following raw data sets are required, for example: for each position of the patient (for the positions face up/face down, left and right lateral position, head or feet first in each case for these positions), two data sets are required for the table direction in each case (a total of 16 possibilities) and two for different voltages for each table direction, i.e., a total of 32 data sets. If further variants are added, such as, for example, different rotational speeds or different integration times, the number of variants have to be multiplied by the 32 data sets.

If the topo protocols are to be changed, corresponding new test data has to be generated. Herein, the patient or phantom positions must match the previous topo positions, otherwise the main scan positions will no longer match.

However, this is only the minimum amount of test data. Usually, there are other variants of parameters that depend on the scan area, such as, for example, pitch (0.35-1.5 with 0.05 steps), rotation (0.25, 0.30. 0.33, 0.50. 1.0), voltage (70 kV-140 kV), current (any current) and many more.

If, for example, tests are to be performed based on images showing an entire human body, a main scan over a range of two meters would typically take 50˜80 seconds. Herein, an image file can be up to 200 GB per scan (i.e., a single data set). For the amount of test data mentioned in the aforementioned example, the required storage capacity could easily be several hundred terabytes if many variants are to be tested.

In addition to image reconstruction, it is also necessary to test system test sequences with real detector raw data. However, without real detector hardware, no images can be generated.

Real raw data depends on tuning tables. If the detector hardware is changed, the images can only be used to a limited extent, since artifacts can occur in the images. This means that older raw data can no longer be used.

To date, a fixed position has been defined for a whole-body phantom. The head was positioned at a specific position, for example, 500 mm. Then, topo scans were performed from 400 mm to 2455 mm thus enabling a 2 m topo scan. Main scans were performed with the same positions.

Each time the scan protocol is changed, the scans are performed again with the whole-body phantom at the same positions and the raw data is recorded. This can easily take several hours if many data sets are required.

For non-existent detector hardware, or for tests irrelevant to customer workflows, testing was performed with simulated raw data. If the detector hardware is changed, all protocols must be renewed since the tuning tables no longer match and artifacts appear on the images.

### SUMMARY

Example embodiments provide a method and an apparatus for testing algorithms or systems for processing, evaluating or reconstructing X-ray-based images, a control facility for an X-ray imaging system and an X-ray imaging system with which the above-described disadvantages are avoided. According to one or more example embodiments, as many test data sets as possible are generated in a short time with a minimum “basic data set”.

### BRIEF DESCRIPTION OF THE DRAWINGS

The invention is explained again in more detail below with reference to the accompany figures and with reference to different exemplary embodiments. Herein, the same components are given identical reference symbols in the different figures. The figures are generally not to scale. The figures show:

FIG. 1 illustrates a rough schematic representation of a CT system according to the prior art,

FIG. 2 illustrates a block diagram of an example of a method according to one or more example embodiments,

FIG. 3 illustrates options for variable parameters of a recording according to one or more example embodiments, and

FIG. 4 illustrates the generation of simulation images according to one or more example embodiments.

### DETAILED DESCRIPTION

A method according to one or more example embodiments is used to test algorithms or systems or systems for processing, evaluating or reconstructing X-ray-based images. It comprises the following steps:
providing at least one working image in the form of a CT image,
generating a number of simulation images from the at least one working image, wherein in each case image values of the working image for image points of the simulation images are summed up along a line,
outputting the simulation images, in particular for testing a module for processing, evaluating or reconstructing images or for training a network with a machine-learning capability.

The method is used to test algorithms or systems for processing, evaluating or reconstructing X-ray-based images. These can, for example, be image reconstruction systems, evaluation systems or image processing systems (embodied as hardware or software). Herein, the method is also suitable for generating training for training a model with a machine-learning capability that is to be used in a corresponding system. The method is, for example, suitable for generating raw CT data with which an image reconstruction system can be tested or that can be reconstructed into CT images in order to test evaluation systems or image processing systems.

First, at least one working image is provided in the form of a CT image. The working image is thus a 3D image of an object, in particular a patient, which has preferably been reconstructed from projection images and is preferably available as a stack of slice images. The working image should have been recorded with an imaging system for which the systems in question are to be tested, but this is not absolutely necessary. If the imaging system in question is not accessible or does not yet exist, a working image can also have been recorded with another imaging system. Here, it is particularly preferable for the basic materials of the object recorded in the working image to be known, for example, bone or soft tissue. It is also particularly preferable for the working image to be available as a dual-energy image or multi-energy image or for a plurality of working images of the same motif which have been recorded with different X-ray energies to be available.

Preferably, a working image shows at least part of the body of a patient, a phantom or an object. Basically, only one image is required. However, there can also be several images; in particular in cases where several patient positions (prone position, lateral position) are to be simulated or projection images with several energies are to be simulated, several CT images with different recording energies can be available. However, there could also be a CT image in which each image point has a vector of values at different energies.

However, for a good understanding of the following steps, it is sufficient to imagine that a working image is provided that represents a human body in three dimensions.

In a next step, a number of simulation images is generated from the at least one working image. These simulation images are preferably projection images. It is possible for only one single simulation image to be generated, for example, if systems of a radiography system are to be tested, or several images, in particular if systems of a CT system or tomosynthesis system are to be tested. In particular, simulation images can be generated in the form of projection images that show the motif of the number of working images from different perspectives and in particular correspond to raw data from a CT system or tomosynthesis system.

Herein, it is important that for each image point of the simulation images, image values of the at least one working image are summed up along a respective line. This can be performed very quickly since summation along a straight line can be calculated very easily and in a time-saving manner with a computer. Herein, it is preferable for there to be a separate line for each image point of a simulation image that passes through the working image and a predetermined detector plane. Herein, the detector plane can be straight or curved and should correspond to the detector plane of the image recording system of which the systems are to be tested. Herein, it is assumed that the image point in question lies exactly at the point of intersection between the line and the detector plane.

Even if an imaging system does not yet exist, its geometric structure is known. In particular, it is known where the X-ray source is located, where the detector is arranged, where the patient bench is to be inserted as intended and which elements exist that can influence the X-ray beam. Before each pass of the method, it is then possible to specify a bundle of lines that intersect the detector plane in a grid (corresponding to image points of the simulation image) and originate from a common point or at least a common surface. This bundle of lines is referred to in the following as a “beam cone”, since it simulates a cone-shaped X-ray beam. The beam cone should have the shape of an X-ray beam that falls from the X-ray source onto the detector and may have been influenced by elements. Since the scanner geometry is known, the shape of a beam cone is always known, even with different beam variations. It should be noted that there can quite possibly be imaging systems that generate multiple cones of X-ray beams, for example, systems with two or more X-ray sources that emit simultaneously. In this case, the lines are simply arranged in the form of corresponding beam cones.

It should also be noted that it is quite possible for scattering or cross-scattering to be taken into account. In this case, lines are added that simulate X-rays from these effects. These lines then add value to the image points of the simulation image that is weighted according to the intensity of this scattered radiation. The calculation methods used for this are generally known.

It is quite possible for the beam cone to have a predetermined intensity profile, for example, a profile in which the beams should be brighter in the center than at the edge. This can be achieved with additional factors that are multiplied by the respective sum of the image values of the working image. These factors can also be used to take into account the intensity of the beam and shadows.

Similarly, it can be taken into account that the detector allows energy resolution and/or has photon-counting properties.

However, an energy-resolved working image would be advantageous for this.

The lines pass through the working image and intersect its image points. It is then possible to calculate the image value of the corresponding image point of the simulation image from the sum of the image values of the intersected image points. However, it should be noted in this regard that it is usually advantageous to normalize this image point. For this purpose, the distance by which the line passes through a voxel could be taken into account or the total length of the line through the working image could be taken into account or averaged over a certain number of voxels. In practice, weighting is preferably carried out with regard to the path length of the beam or the line path in the motif. The image values can in particular represent linear attenuation coefficients μ (usually μwater) for each image point of the simulation image. The total attenuation is then the sum of all μ*path lengths of the simulated beam (line) in the voxel.

To generate a plurality of simulation images in the form of raw CT data, the beam cone be rotated together with the detector plane about the motif of the working image and a simulation image can be created at different rotation angles in each case.

Accordingly, the feed of a patient bench can be simulated. In this regard, the working image is simply shifted relative to the beam cone and the detector plane.

It should also be noted that the working images can quite possibly be manipulated for different simulation images. For example, it is possible to add an element from other CT images. For example, a working image can be provided with a diseased area or a healthy bone can be replaced by a broken bone. In general, it is preferable to incorporate limited and individually documented clinical pictures from other CT images into the working images. However, organs can also be exchanged with other organs from other 3D image volumes. Here, it is advantageous to always use images or image sections of real motifs so that the simulation images are based on real images. However, it is also quite possible to use an image of a real motif and to artificially change parts of the image content.

After the simulation images have been generated, they are output. Herein, they can be used directly to test a module for processing, evaluating or reconstructing images or for training a network with a machine-learning capability or they can be saved. However, it is preferable to use them immediately since the aim is to use as little storage space with test data (sets of simulation images), and to generate these quickly as required. However, if it is clear from the outset that sets of simulation images are to be used multiple times, storage could quite possibly be advantageous.

An apparatus according to one or more example embodiments is used to test algorithms or systems for processing, evaluating or reconstructing X-ray-based images. It comprises the following components:
a data interface designed to receive at least one working image in the form of a CT image,
a simulation unit designed to generate a number of simulation images from the at least one working image, wherein in each case image values of the working image for image points of the simulation images are summed up along a line,
a data interface designed to output the simulation images, in particular for testing a module for processing, evaluating or reconstructing images or for training a network with a machine-learning capability.

The function of the components of the apparatus has already been described above. The apparatus is preferably designed to execute a method according to one or more example embodiments.

One or more example embodiments enables the use of only one input file (the number of working images) for a plurality of different (existing and non-existing) imaging systems in future and enables test data sets tailored to a respective situation to be generated quickly and easily. Once a detector geometry has been defined, this procedure can generate simulation images without genuine hardware for a desired imaging system, preferably as simulated raw data. The only input file required is a number of essentially arbitrary 3D image volumes, wherein each working image should contain the desired motif. These can, for example, be DICOM images. The working images are therefore essentially used as a virtual patient.

The method can easily simulate different rotational speeds, integration times, X-ray voltages, beam currents, table speeds, gantry tilt angles or other parameters and generate corresponding simulation images. Based on known system architecture, new raw data can be created for imaging systems that do not yet exist. Preliminary investigations can therefore be performed without hardware that actually exists. The use of the same working images has the effect that simulations of topo scans, spiral scans, zigzag scans, sequence scans and other scans always start from the same initial data. This ensures that the training data always matches. Instead of several thousand files, the method only requires a few working images, for example, four, because in this way every typical patient position (face up/down and left and right lateral positions) is available. It is preferable for the working images to contain voltage information. This enables the voltage effects in the raw data to be converted. If no images exist for a desired table position, the method can generate raw “air” data. This corresponds to reality since, if there is no patient bench or no patient in the recording area of a CT system, raw “air” data is also generated there. Simulation images of air recordings can be used for system tests, for example, for calibration workflows.

AI algorithms can be validated more easily since the working images are defined and known. If working images of tuning phantoms exist, system tuning steps can also be performed successfully. The method according to one or more example embodiments can be used to test very detailed and deeper system functions. This procedure enables system experience at a real system level.

A control facility according to one or more example embodiments for an X-ray imaging system comprises an apparatus according to one or more example embodiments and/or is designed for performing a method according to one or more example embodiments.

An X-ray imaging system according to one or more example embodiments comprises a control facility according to one or more example embodiments.

One or more example embodiments can in particular be realized in the form of a computer unit with suitable software. For this purpose, the computer unit can, for example, have one or more interworking microprocessors or the like. In particular, it can be realized in the form of suitable software program components in the computer unit. A largely software-based realization has the advantage that even computer units used to date can easily be retrofitted by a software or firmware update in order to operate in the manner according to one or more example embodiments. In this respect, the object is also achieved by a corresponding computer program product with a computer program, which can be loaded directly into a storage facility of a computer unit, with program sections for executing all steps of the method according to one or more example embodiments when the program is executed in the computer unit. In addition to the computer program, such a computer program product can optionally comprise additional items such as, for example, documentation and/or additional components, including hardware-components, such as, for example, hardware keys (dongles, etc.) for using the software.

For transport to the computer unit and/or for storage on or in the computer unit, it is possible to use a computer-readable medium, for example a memory stick, hard disk or other transportable or permanently installed data carrier on which the program sections of the computer program that can be read and executed by a computer unit are stored.

Further, particularly advantageous embodiments and developments of the invention emerge from the dependent claims and the following description, wherein the claims in one claim category can also be developed analogously to the claims and description parts for another claim category and in particular individual features of different exemplary embodiments or variants can also be combined to form new different exemplary embodiments or variants.

Preferably, a working image shows a patient in the supine position, in the prone position, in the right lateral position, in the left lateral position or in the standing position. There is preferably a plurality of working images showing different positions of the patient. Preferably, the patient's head or feet are aligned with the gantry. Preferably, there is a plurality of working images of the same patient recorded at different positions of the patient. Here, “patient” means humans, animals or objects of which recordings can be taken in a prone, supine lateral position.

Preferably, there is a plurality of working images of the same motif which have been recorded at different recording energies. These are preferably dual-energy or multi-energy recordings.

According to a preferred embodiment of the method, to generate a simulation image, a plurality of lines is defined starting from its image points to a predetermined position of an X-ray source. These lines are then used to add the image values of the working image for the respective image point. As already explained above, it is preferable to define a beam cone (bundle of lines) for this purpose.

Herein, it is preferable for a plurality of simulation images to be created for different positions of the X-ray source which lie on a circular or spiral path (with the working image unchanged or with the working image shifted laterally). For this purpose, the beam cone can preferably be rotated on a predetermined path in space about the respective working image and, herein, the image points of the simulation images can be calculated for multiple positions of the beam cone.

According to a preferred embodiment of the method, the positions of the X-ray source are defined at (in particular constant) angular intervals which have been ascertained according to a predetermined rotation time of the gantry and a predetermined integration time of the measurements. This simulates the recording of projection images in constant angular sections.

According to a preferred embodiment of the method, a plurality of positions is defined based on a predetermined feed of a patient bench, a predetermined rotational speed of an X-ray source and predetermined recording times. This, for example, enables spiral or zigzag scans to be simulated. Herein, it is preferable that:
a spiral scan is simulated by selecting positions corresponding to a constant rotation about a moving patient bench, or
a topo scan is simulated by selecting positions corresponding to a stationary X-ray source above (preferably at an angle of 0°) or next to (preferably at an angle of 90°) a moving patient bench, or
a sequence scan is simulated by selecting positions on a plurality of circular paths which in each case has a distance of a predetermined detector width along the patient bench, or
a zigzag scan is simulated by selecting positions corresponding to a patient bench moving back and forth and an X-ray source rotating (in particular at a constant speed). This enables, for example, a heart, to be scanned several times in order to measure the temporal course of a contrast agent or a movement.

According to a preferred embodiment of the method, the lines are arranged such that they form a cone (said beam cone). The direction of its normal to the transverse plane of the working image (A) is preferably inclined by a predetermined angle. Herein, for the inclination of the angle to simulate the tilt of a gantry.

It is preferable that, in one embodiment of the method, the image points of the simulation image lie on a flat or curved surface. The shape of the simulation image should correspond to the shape of the inner surface (facing the X-ray source) of the detector of the real or desired imaging system. Herein, it is preferable that, in the case of a curvature of the simulation image (the detector plane), the radius of curvature corresponds to a defined distance to an X-ray source. Flat or curved detectors for example, are often used In a CT system. Herein, often a curved detector with a constant distance between each detector point and the X-ray source is used.

It is preferable that collimation of an X-ray beam is simulated by assigning a predetermined value reduction to image points at the edge of a projection image. This can in particular take place as described above with the beam cone and the factors.

Each line is assigned a factor by which sum of the image values is multiplied. Small factors correspond to a lower beam intensity. If the beam is collimated such that part of the “detector” would no longer see anything, the factors of the lines can quite possibly also have a value of zero.

It is preferable that, in one embodiment of the method, the definition of the lines is based on a predetermined beam direction of the X-ray source. Herein, it is preferable that the weighting of the individual summed values is defined in this regard. This, for example, simulates beams originating from an X-ray source with a spring focus that deflects a beam according to a predetermined beam direction. The intensity profile is preferably also simulated with the beam cone and the factors by reproducing the intensity profile using the factors.

According to a preferred embodiment of the method, prior to generating the number of simulation images, image values of the working image are modified according to a function that simulates recording at a different recording energy. Herein, it is preferable for the working image to have been recorded with a plurality of recording energies and for the function of the difference between the image values in a voxel of the working image to depend on the difference of the respective image value at the different recording energies. If the distribution of basic materials in the recorded image is known, the absorption spectra of these basic materials can also be used for simulation. Herein, an energy is predetermined for creating a simulation image and, during addition, the image values are weighted according to the absorption spectra of the basic materials determined in the image points.

According to a preferred embodiment of the method, prior to generating the number of simulation images, image values of the working image are modified according to a function that simulates recording at a predetermined radiation flux or collimator filter or a predetermined aperture. This function is known in advance or defined by a user.

Additional image noise can be added to the simulation image if this is appropriate for the detector of the imaging system to be simulated.

According to a preferred embodiment of the method, image information is added to the provided working images, preferably from other X-ray-based images, which alters the depicted patient, wherein in particular a pathology or an implant is added thereto and/or an organ is replaced by a corresponding organ from another CT image. This enables the working images to be altered in a defined manner such that they display predetermined characteristics. In particular, this is advantageous for generating training data. Thus, a defined alteration can be added to a previously known working image and knowledge of this change can be used as ground truth for training. For example, a pathology and then a model with a machine-learning capability can be specifically trained on this type of pathology.

Preferably, components of the invention are available as a “cloud service”. Such a cloud service is used to process data, in particular via artificial intelligence, but can also be a service based on conventional algorithms or a service in which evaluation is performed by humans in the background. In general, a cloud service (hereinafter, also “cloud” for short) is an IT infrastructure in which, for example, storage space or computing power and/or application software is made available via a network for example. Herein, communication between the user and the cloud takes place via data interfaces and/or data transmission protocols. In the present case, it is particularly preferable for the cloud service to provide both computing power and application software.

In a preferred method, data obtained in the context of one or more example embodiments is provided via the network to the cloud service. This comprises a computing system that does not usually include the user's local computer. Herein, the method can be realized via a command constellation in a network. The data in the cloud is later sent back via the network to the user's local computer.

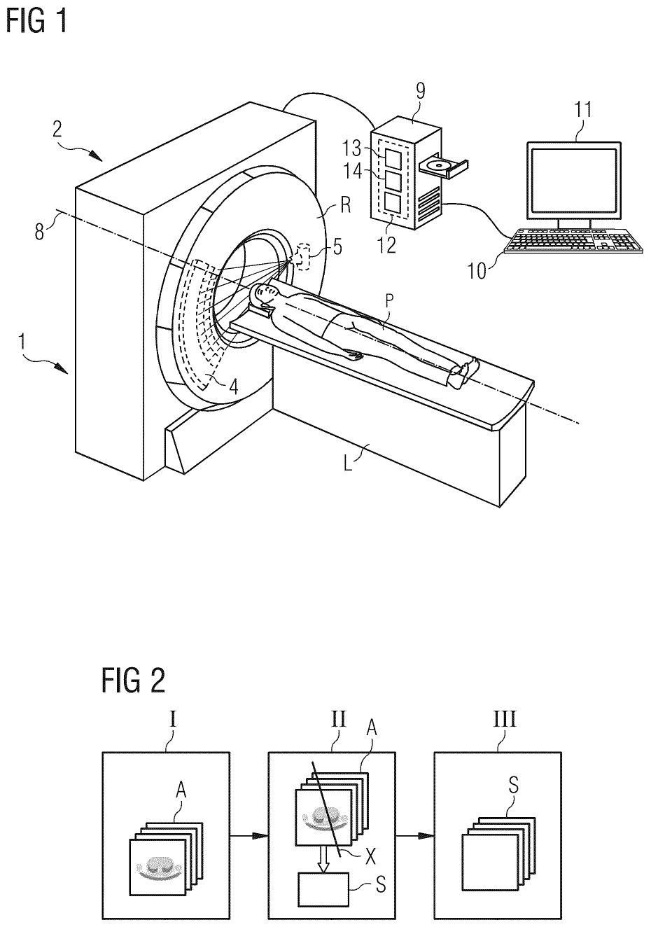

FIG. 1 shows a computed tomography system (CT system) 1 with a radiation detector 4 and an X-ray source 5. The X-ray source 5 is embodied to expose the radiation detector 4 to X-rays. The CT system 1 shown comprises a gantry 2 with a rotor R. The rotor R comprises the X-ray source 5 and the radiation detector 4.

The rotor R can rotate about the axis of rotation 8. The patient P, is supported on the patient bench L and can be moved along the axis of rotation 8 through the gantry 2. The computing unit 9 is provided to control the CT system 1 and/or to generate an image data set based on signals detected by the radiation detector 4.

A (raw) X-ray image data set of the patient P is usually recorded from a plurality of angular directions via the radiation detector 4. Subsequently, a (final) image data set can be reconstructed based on the (raw) X-ray image data set via a mathematical method, for example comprising filtered back projection or an iterative reconstruction method.

Here, the computing unit 9 serves as a control facility (also referred to as a controller) 9 for controlling the CT system 1. This computing unit 9 is connected to an input facility 10 and an output facility 11. The input facility 10 and the output facility 11 can, for example, enable interaction by a user or the representation of a generated image data set B.

The computing unit 9 comprises an apparatus 12 for testing algorithms or systems for processing, evaluating or reconstructing X-ray-based images. The apparatus 12 comprises a data interface 13 and a simulation unit 14.

The data interface 13 serves to receive at least one working image A in the form of a CT image and to output simulation images S, in particular for testing a module for processing, evaluating or reconstructing images or for training a network with a machine-learning capability.

The simulation unit 14 serves to generate a number of simulation images S from the at least one working image A, wherein in each case image values of the working image A for image points of the simulation images S are summed up along a line X. The generation is explained in more detail in FIG. 4.

FIG. 2 shows a method for testing algorithms or systems for processing, evaluating or reconstructing X-ray-based images.

In step I, a working image A is provided in the form of a CT image. Here, it is indicated that the working image A is a three-dimensional image in the form of a stack of slice images. It represents the body of a patient P and was, for example, recorded by the CT system shown in FIG. 1.

In step II, a number of simulation images S is generated from the working image A in the form of projection images, wherein in each case image values of the working image A for image points of the simulation images S are summed up along a line X (see FIG. 4).

In step III, the simulation images S are output, in particular for testing a module for processing, evaluating or reconstructing images or for training a network with a machine-learning capability.

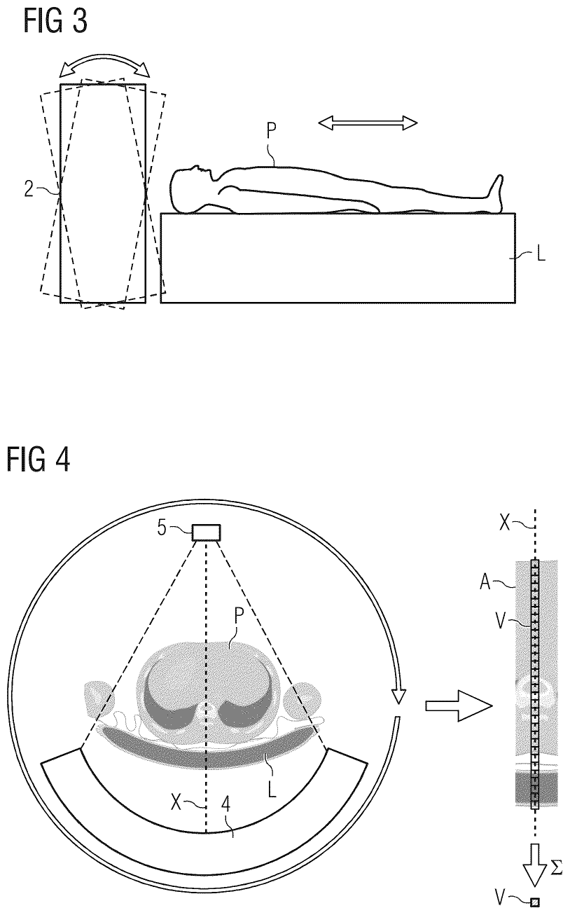

FIG. 3 shows options for variable parameters of a recording. The left-hand side shows the gantry 2 of the CT system in FIG. 1; this can be tilted up to the dashed positions according to the bent arrow. The right-hand side shows a patient bench that can be moved back and forth at different speeds along the straight arrow. Further parameters relate to the beam, the rotation of the rotator R and the position of the patient P.

FIG. 4 shows the generation of simulation images S. Here, it is indicated on the left that the patient P is transilluminated by a beam cone (dashed lines) emanating from the X-ray source 5 and the beam is captured by the radiation detector and its intensity measured. In the event of the patient P being the image information of the working image A and the beam cone being a bundle of lines X, the left-hand image would also show the prerequisite for calculating the image values of a simulation image B.

Let us assume that the value of the image point V of the simulation image through which the vertical line X extends at the detector 4 is to be calculated. Then, as shown in the left-hand image, the image values of the image points V of the working image A would be summed up along the course of line X and preferably also normalized. As shown in the left-hand image, all voxels V on the line add up to a voxel V of the simulation image (at the bottom).

Finally, reference is made once again to the fact that example embodiment described above in detail merely entails different exemplary embodiments which the person skilled in the art can modify in a wide variety of ways without departing from the scope of the invention. Furthermore, the use of the indefinite articles “a” or “an” does not preclude the possibility that the features in question can also be present on a multiple basis. Likewise, terms such as “unit” do not preclude the possibility that the components in question consist of a plurality of interacting sub-components, which could also be spatially distributed. The term “a number” should be read as “at least one”. Independent of the grammatical term usage, individuals with male, female or other gender identities are included within the term.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,”etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for testing algorithms or systems for processing, evaluating or reconstructing X-ray-based images, the method comprising:
providing at least one working image, the at least one working image including a CT image;
generating a number of simulation images from the at least one working image, wherein for each image point of the simulation images, image values of the at least one working image are summed along a respective line; and
outputting the simulation images.

2. The method of claim 1, wherein
at least one of the at least one working image shows a patient in a supine position, in a prone position, in a right lateral position, in a left lateral position or in a standing position.

3. The method of claim 1, wherein the generating includes defining a plurality of lines starting from respective image points to a predetermined position of an X-ray source and the lines are used to add the image values of the at least one working image for the respective image point.

4. The method of claim 3, wherein at least one of
the positions of the X-ray source are defined at angular intervals which are ascertained according to a predetermined rotation time of a gantry and a predetermined integration time of measurements, or
a plurality of positions is defined based on a predetermined feed of a patient bench, a predetermined rotational speed of an X-ray source and predetermined recording times.

5. The method of claim 3, wherein the lines form a cone, and a direction of a normal of the cone to a transverse plane of the at least one working image is inclined by a predetermined angle.

6. The method of claim 3, wherein the image points of the simulation image lie on a flat or curved surface and collimation is simulated by assigning a predetermined value reduction to image points at an edge of a projection image.

7. The method of claim 3, wherein the definition of the lines is based on a predetermined beam direction of the X-ray source.

8. The method of claim 1, wherein, prior to generating the number of simulation images, image values of the at least one working image are modified according to a function that simulates recording at a different recording energy.

9. The method of claim 1, wherein, prior to generating the number of simulation images, image values of the at least one working image are modified according to a function that simulates recording at a predetermined radiation flux, a collimator filter, or a predetermined aperture.

10. The method of claim 1, wherein image information is added to the provided at least one working image, which alters a depicted patient.

11. An apparatus configured to test algorithms or systems for processing, evaluating or reconstructing X-ray-based images, the apparatus comprising:
a data interface configured to receive at least one working image, the at least one working image including a CT image;
a simulation unit configured to generate a number of simulation images from the at least one working image, wherein for each image point of the simulation images, image values of the at least one working image are summed along a respective line; and
a data interface configured to output the simulation images.

12. A controller for an X-ray imaging system, the controller comprising:
the apparatus of claim 11.

13. An X-ray imaging system comprising:
the controller of claim 12.

14. A non-transitory computer program product comprising instructions which, when executed by a computer, cause the computer to perform the method of claim 1.

15. A computer-readable storage medium comprising instructions which, when executed by a computer, cause the computer to perform the method of claim 1.

16. The method of claim 1, wherein the outputting outputs the simulation images for testing a module for processing, evaluating or reconstructing images or for training a network with a machine-learning capability.

17. The method of claim 2, wherein at least one of
a head or feet of the patient are aligned with a gantry, or
there is a plurality of working images of a same motif which have been recorded at different recording energies.

18. The method of claim 3, wherein a plurality of simulation images is created for different positions of the X-ray source which lie on a circular or spiral path.

19. The method of claim 4, wherein:
a spiral scan is simulated by selecting positions corresponding to a constant rotation around a moving patient bench,
a topo scan is simulated by selecting positions corresponding to a stationary X-ray source above or next to a moving patient bench,
a sequence scan is simulated by selecting positions on a plurality of circular paths, each of the circular paths has a distance along the patient bench, or
a zigzag scan is simulated by selecting positions corresponding to a patient bench moving back and forth and a rotating X-ray source.

20. The method of claim 5, wherein the inclination of the angle simulates a tilt of a gantry.
